(19) United States
(12) Patent Application Publication
(10) Pub. No.: US 2025/0222147 A1
(43) Pub. Date: Jul. 10, 2025

(54) IMPROVED STERILISATION AUTOCLAVE

(71) Applicant: FEDEGARI AUTOCLAVI SPA, Pavia (IT)

(72) Inventors: Paolo FEDEGARI, Celerina/Schlarigna (CH); Giuseppe FEDEGARI, Pavia (IT)

(73) Assignee: FEDEGARI AUTOCLAVI SPA, Pavia (IT)

(21) Appl. No.: 18/854,571

(22) PCT Filed: Apr. 21, 2023
(86) PCT No.: PCT/IB2023/054093

§ 371 Date: Oct. 7, 2024

(30) Foreign Application Priority Data
Apr. 22, 2022 (IT) 102022000007991

(51) Int. Cl. A61L 2/07

(52) U.S. Cl. CPC A61L 2/07; A61L 2202/121; A61L 2202/122; A61L 2202/15

(57) ABSTRACT

Sterilization autoclave (1) comprising: a sterilisation chamber (2); a removable door; a plate (3) having two opposing sides with a greater extension and positioned in the sterilisation chamber (2) in proximity to a first wall (2a) of said sterilisation chamber (2), said plate (3) being positioned so as to have a first side (2a) of said two opposing sides facing said first wall (2a) and one or more nozzles (4a, 4b) configured to introduce a flow of sterilising fluid into the sterilisation chamber (2), wherein: the plate (3) identifies with said first wall (2a) an open gap (6) having a predefined separate secondary volume and in fluid communication with the remaining volume of the sterilisation chamber (2), said one or more nozzles (4a, 4b) are positioned to introduce a flow of sterilising fluid directly into said open gap (6), so that said flow of sterilising fluid introduced into the gap (6) creates a suction dragging effect into said secondary volume of fluid present inside the remaining volume of the sterilisation chamber (2) ensuring fluid circulation in said sterilisation chamber (2).

### TECHNICAL FIELD

The present invention relates to a sterilisation autoclave and method for sterilising objects and substances in the medical or chemical-pharmaceutical field.

In particular, the products to be sterilised are containers of various types, in glass, metals or other materials, indicatively for pharmaceutical, chemical and medical use.

### PRIOR ART

With particular reference to the medical or chemical/pharmaceutical field, it is common practice to sterilise every medical device. This operation takes place with different methods according to the type of materials and devices to be sterilised and is codified by strict standards that aim at health protection and guarantee the safety of patients, hospital operators and the environment.

A commonly used sterilisation process envisages the exposure of the objects to substances to be sterilised at temperatures in the range between +110° C. and +140° C. through superheated steam, along with a possible baric treatment at pressures greater than or equal to ambient pressure (e.g. between −2 and 6 bar, preferably between 0 and 3 bar absolute), inside so-called stainless steel autoclaves.

To facilitate the loading and unloading operations of the objects or substances to be sterilised, stainless steel pallets are provided which allow the simultaneous stacking and treatment (insertion in the autoclave, sterilisation and removal from the autoclave) of a plurality of objects and substances subjected to the same sterilisation cycle.

To date, the mostly used process for the sterilisation of containers, for example in glass or metal, for pharmaceutical use consists of a sterilisation treatment with water vapour, for example, saturated and/or superheated at a constant predetermined temperature that lasts uninterruptedly for a predefined exposure time.

The sterilisation process with water vapour is very simple and extremely effective in terms of decontamination.

The typical operation cycle of the sterilisation autoclaves usually involves the following steps:
1. vacuum pump depressurisation;
2. one or more dynamic pulsations;
3. heating;
4. sterilisation;
5. cooling;
6. vacuum pump depressurisation.

Autoclaves of known type use steam spreading pipes for the introduction of steam inside the sterilisation chamber. The use of a modulating valve allows the steam inlet to be opened, closed and/or modulated during the various steps of operation of the sterilisation autoclave.

In the autoclaves of known type, moving components, such as fans, are used to carry out circulation of air and steam inside the sterilisation chamber.

Within the sterilisation autoclaves, more or less complex fluid-dynamic phenomena occur that involve physical mechanisms of different nature, which distinguish the specific process of the machine.

In particular, there are exchanges of matter such as condensation and evaporation of the fluid and thermal exchanges such as conduction, convection and heat radiation.

A problem with the autoclaves of known type lies in the slow diffusion and non-uniformity in the distribution of the water vapour within the sterilisation chamber.

Another problem with the autoclaves of known type is that the optimal temperature distribution for the sterilisation is initially slow and little uniform.

Another problem with the autoclaves of known type is the high energy consumption required for the operation of the autoclave during the steps described above.

A further problem of the autoclaves of known type lies in the possible formation of liquid film in the internal walls of the sterilisation chamber.

Another problem with the autoclaves of known type is the possible formation of water vapour stagnation zones.

A further problem of the autoclaves of known type lies in the possible formation of condensed water vapour at the products to be treated, which can even wet them.

Another problem with the autoclaves of known type is that they require the use of moving components to optimize the circulation of air and steam inside the sterilisation chamber.

A further problem of the autoclaves of known type lies in the fact that they require a certain interval of time to reach thermal uniformity over the entire internal volume of the autoclave.

Another problem with the autoclaves of known type lies in the fact that they do not allow to control the mass transfer phenomenon caused by the passage of state of the water vapour, in particular, to effectively control the field of motion and the actual amount of condensate.

### OBJECT OF THE INVENTION

In this context, the object of the present invention is therefore to propose an improved sterilisation autoclave that allows to overcome the drawbacks indicated above with reference to the prior art.

Within this general object, a particular object of the present invention is to allow a rapid diffusion and uniformity in the distribution of the water vapour within the sterilisation chamber.

Another object of the present invention is to allow a rapid and uniform temperature distribution within the sterilisation chamber.

A further object of the present invention is to allow a low consumption of energy required for the operation of the autoclave during the steps described above.

Another object of the present invention is to prevent or minimize the formation of water vapour stagnation zones within the sterilisation chamber.

A further object of the present invention is to prevent or minimize the formation of condensed water vapour at the products to be treated.

Another object of the present invention is to carry out a circulation of air and steam inside the sterilisation chamber without the use of moving components.

A further object of the present invention is to optimize the process of circulation of air and steam inside the sterilisation chamber, also for the heating and cooling steps.

Another object of the present invention is to allow to have a thermal uniformity over the entire internal volume of the autoclave.

Not least object of the present invention is to allow to control the mass transfer phenomenon caused by the passage of state of the water vapour, in particular, to effectively control the field of motion and the effective amount of condensate.

### BRIEF DESCRIPTION OF THE DRAWINGS

Additional features and advantages will become more apparent from the description of preferred but non-exclusive embodiments of a sterilisation or decontamination autoclave that are illustrated by way of indicative and non-limiting example in the appended drawings, in which:

FIG. 1 shows a detail in perspective view of a sterilisation chamber with quadrangular section;

FIG. 2 shows a front section of FIG. 1;

FIGS. 3 to 8 show a side section of a sterilisation chamber according to various embodiments of the present invention;

FIG. 9 shows an additional heating plate applicable to the sections from FIG. 3 up to FIG. 8;

FIG. 10 shows a partial cross-section of a further embodiment;

FIG. 11 shows a heating plate for the sterilisation chamber of FIGS. 1 to 10;

FIG. 11A shows a detail of the heating plate of FIG. 11.

### DETAILED DESCRIPTION OF PREFERRED EMBODIMENTS OF THE INVENTION

In a first aspect, the present invention describes a sterilisation autoclave or machine.

With reference to FIG. 1, a schematic view of a sterilisation or decontamination chamber 2 inside which the products to be sterilised are arranged is shown.

The sterilisation chamber 2 is contained within a sterilisation or decontamination autoclave 1.

The sterilisation chamber 2 is closed and is intended to contain the products to be sterilised in its inside, the chamber 2 identifies an internal compartment and is provided with an opening that identifies a passage for inserting/collecting the products to be sterilised.

A removable door associated when closed with said opening for passing from an open position, distanced from said opening, to a closed position in which it guarantees the hermetic closure of said passage of said sterilisation chamber 2.

In use, the chamber 2 is hermetically closed by the door and the sterilising agent (e.g., steam and/or sterilising gas) is fed therein as described below.

The autoclave can have a cylindrical, quadrangular or any other shape.

The sterilisation chamber 2 can extend horizontally or vertically (like, for example, in the small mechanics used in laboratories).

In the present description, the vertical axis of the sterilisation machine 1 means an axis perpendicular to the floor on which the autoclave rests during its operation.

The sterilisation machine comprises at least one plate 3, for heating/cooling by means of process fluid, having two opposing sides with a greater extension and positioned in said sterilisation chamber 2 in proximity to a first wall 2a of said sterilisation chamber 2.

The plate is heated or cooled with a fluid, e.g., steam, air, or water.

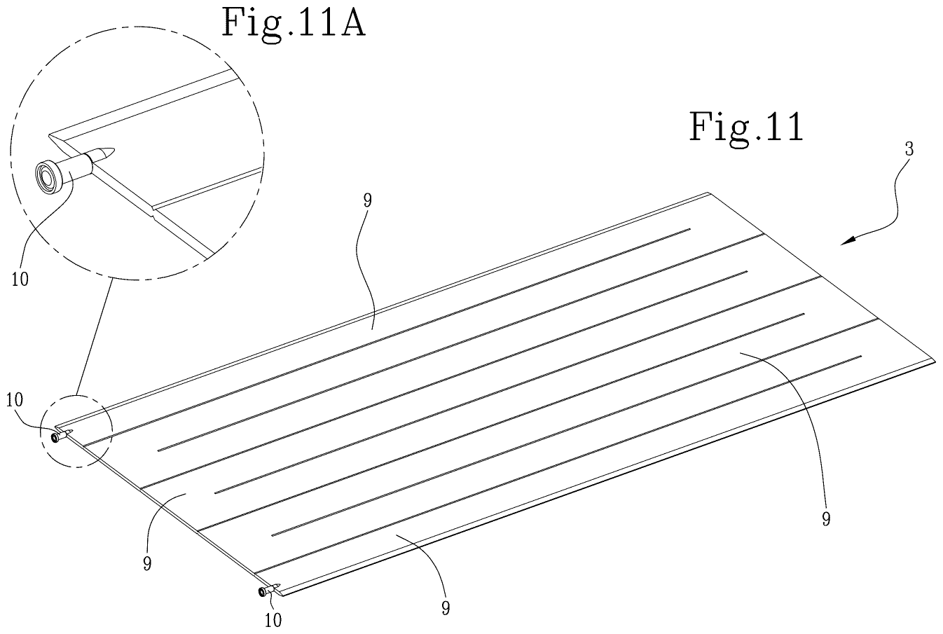

In accordance with the embodiment of FIG. 11, the heating plate 3 comprises a coil 9 which identifies a tubular passage for a working fluid (a liquid, a vapour or a gas), for heating or cooling.

Preferably, the heating plate 3 is shaped so as to identify the aforementioned coil 9 therein.

The coil 9 allows to ensure a heat exchange between the fluid that flows inside it and the sterilisation fluid that is present inside the sterilisation chamber and is in contact with the heating plate 3.

The geometry and the diameter of the coil 9 may vary depending on the specific technical requirements to be met.

As shown in the enlarged detail in FIG. 11A, the coil 9 comprises an inlet end 10 and an outlet end 10 positioned at opposing ends of the coil 9.

In accordance with one embodiment, the coil of the heating plate 3 consists of an electric coil. In this case, the heating plates 3 are heated by the electrical resistors forming the coil. In particular, in this case the heating means converts electrical energy into thermal energy which is irradiated inside the sterilisation chamber 2.

Optionally, it is possible to provide heating plates 3 comprising both the coil 9 with tubular passage for a fluid and the electric coil described above.

The plates 3, 3a, 3b, 3c, 3d, 3e, 3f, 3g have the following purposes:
1. To heat the mixed fluid present inside the gap 6;
2. To heat the internal wall 2a of the autoclave by irradiation;
3. To heat the rest of the fluid by convection and the load present inside chamber 2 by irradiation.

The plates 3 are also used in order to:
pre-heat the load present inside the autoclave (in the first step, prior to sterilisation) in order to reduce condensates;
dry the load once the sterilisation cycle is finished;
cool the load prior to removal from the sterilisation chamber.

The plate 3 is positioned so as to have a first side of said two opposing sides facing said first wall 2a and comprises one or more nozzles 4a, 4b configured to introduce a flow of sterilising fluid, taken from the outside, into said sterilisation chamber 2.

The plate 3 identifies with the first wall an open gap 6 having a predefined separate secondary volume and in fluid communication with the remaining volume of the sterilisation chamber 2.

Preferably, the gap 6 is open at least at one or both of the lower and upper ends of each plate 3.

One or more nozzles 4a, 4b are positioned to introduce a flow of sterilising fluid directly into the open gap 6, so that the flow of sterilising fluid introduced into the gap 6 creates a suction dragging effect into said secondary volume of fluid present inside the remaining volume of the sterilisation chamber 2 ensuring fluid circulation in said sterilisation chamber 2.

The rapid introduction through the nozzles 4a, 4b of a hot flow of sterilising fluid, introduced into the gap 6, also entails the technical effect of heating, rapidly together with the plate 3, 3a-3g, one of the two opposing sides of the autoclave.

Heating the plate 3 allows heat to be radiated directly to the remaining volume of the sterilisation chamber (containing the products to be sterilised) and greatly reduces the amount of possible condensate that could form inside the chamber 2.

The sterilising fluid used may be air or steam or a mixture of air and steam, depending on the needs. The pressure upstream of the nozzle 4a, 4b will be that of the supply circuit of the machine (e.g. 6 barg for the air and 3.5 barg for the steam).

By exploiting the open gap 6 formed by the plates inside the chamber 2 and the position of the nozzles 4a, 4b to carry out circulation of the flow of fluid in the chamber, the Venturi effect in the channel 6 between the plates 3 and the internal part of the chamber 2 is intensified as much as possible.

This phenomenon is similar to that of the mixing ejectors.

The ejectors are formed by two bodies, a nozzle 4a, 4b that is responsible for the introduction of the fluid at high flow rate and speed and by a Venturi-like body, the gap 6, which serves to mix the two flows and to suck the low pressure fluid present in the remaining volume.

The nozzle 4a, 4b of each ejector introduces steam into the chamber 2 while the external body will be obtained from the shape of the plate. Therefore, the assembly of nozzle 4a, 4b plus plates 3 is a single body to form the ejector.

The nozzles 4a, 4b of the ejectors can be connected at the inlet to the outlet of an external steam generator, to a steam supply system, to the compressed, filtered and conditioned air supply (e.g. with exchanger for the heating thereof).

Each ejector has a first inlet for a high pressure primary fluid (steam), a second inlet for a low pressure secondary fluid, a mixing chamber and an outlet, towards the inside of the sterilisation chamber 2, of the mixed primary and secondary fluids.

The diameter of the nozzles 4a, 4b is sized according to the flow rate and pressure conditions in the chamber that depend on the size of the machine and therefore of the chamber.

Preferably, the diameter of the inlet nozzle 4a, 4b is comprised between 10 mm and 20 mm, advantageously, it is equal to 15 mm.

The plates 3, 3a-3g, are heatable to a temperature comprised between 110° C. and 140° C. by circulating the flow of sterilising fluid within the gap 6 (e.g., steam).

Preferably, the plates 3 are heatable to a temperature of 400 Kelvin (126° C.).

Advantageously, inside the sterilisation chamber 2 there are two plates 3 parallel to one another, positioned at a distance from respective opposite walls of the sterilisation chamber 2, each plate 3 defining an open gap 6, in which there are one or more nozzles 4a, 4b.

The plates 3 are preferably made of stainless steel and positioned on the opposite walls of the sterilisation chamber 2, parallel to the loading direction. To favour heat exchange phenomena, they can also extend on the upper part of the autoclave 1.

The shape is optimized to favour the flow of sterilising fluid inside the gap 6 that is formed between the internal wall of the autoclave and the plate 3 itself. The distance between plate 3 and the internal wall of the autoclave 1 is comprised between 10 and 100 mm and must be optimized according to the conditions of transport of the steam or of the air through nozzles 4a, 4b.

Preferably the distance between plate and internal wall of chamber 2 is between 30 mm and 50 mm

The heating fluid passing through each plate 3 can be steam or air with temperature approximately comprised between 110° C. and 140° C. depending on the sterilisation conditions and the step of the cycle. The plates 3 can also serve as a cooling system for the autoclave 1 and of the load.

Various shapes and configurations of plates 3 are illustrated in FIGS. 3 to 10 according to the present invention.

A substantially flat plate 3 is illustrated in FIG. 3.

In FIG. 4 the plate is slightly concave on the side towards the inside of chamber 2 (convex towards a near wall of the chamber 2).

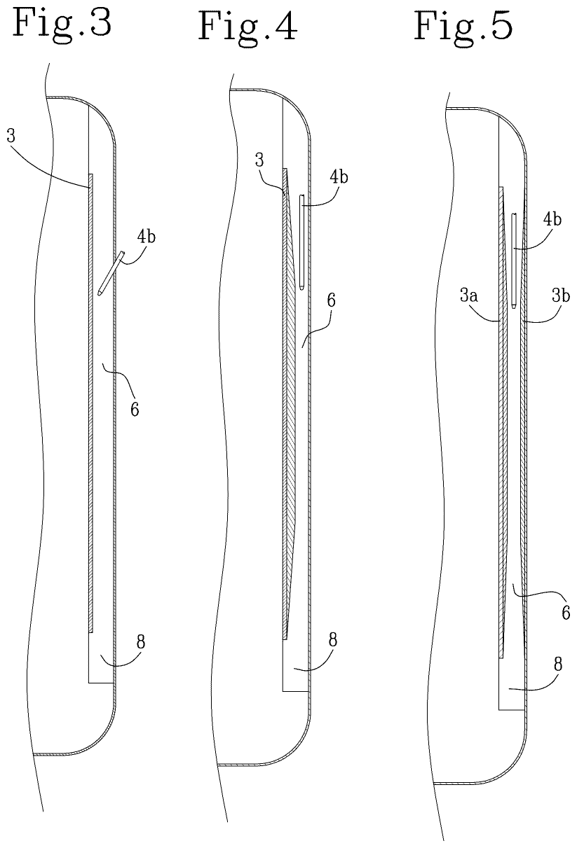

In FIG. 5, there are two plates 3a, 3b, a substantially flat plate 3b close to an internal wall of the chamber 2 and a plate 3a distanced from the plate 3b.

FIG. 8 shows a plate 3c, 3d, 3e consisting of three flat elements, slightly shifted with respect to the vertical longitudinal axis, so as to define one or more passages for the flow of fluid between the secondary volume of the gap 6 and the remaining volume of the chamber 2.

FIG. 7 shows a variant of the plate 3c, 3d, 3e of FIG. 8, in which at least two elements 3d and 3e are slightly inclined with respect to the vertical axis.

The plates (coils) arranged as shown in FIG. 7 or 8 also assume the function of baffles and allow a homogeneous distribution of the flow of sterilising gas (hot/cold steam or air) to different sterilisation chamber sections to optimize the heat exchange among the surfaces.

The combination of ejector and plates leads to an efficient distribution of the fluid (steam or air) inside the autoclave.

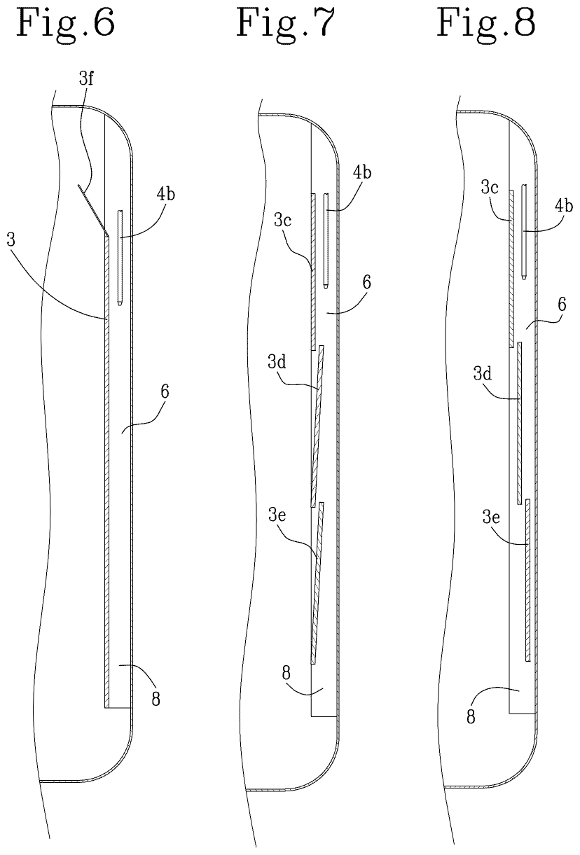

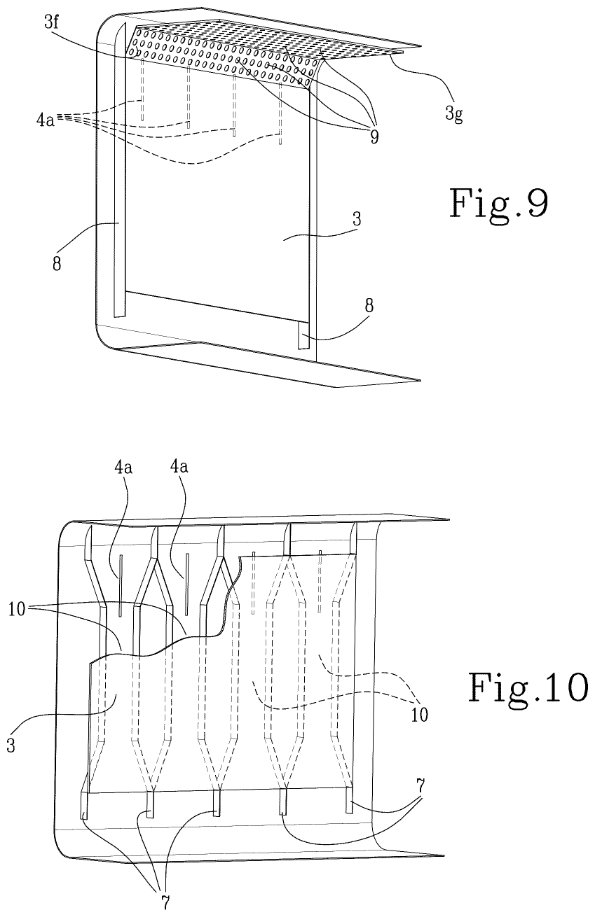

In FIG. 6 a variant is shown in which the upper open end of the plate 3 comprises a perforated deflecting baffle plate 3f, slightly inclined with respect to the vertical axis of the plate 3 (see also FIG. 9).

FIG. 9 shows an embodiment in which the plate 3, 3b comprises at one end a perforated substantially horizontal plate 3g (i.e. perpendicular to the opposing sides of the plate 3).

The perforated horizontal plate 3g can be applied directly to the upper end of the vertical plate 3 or it can be applied as an appendage of the oblique perforated deflecting baffle plate 3f of FIG. 6.

In other words, FIG. 9 represents an additional heating plate 3g placed in the upper part of the chamber 2 and has the purpose of limiting any localized condensation phenomena.

FIG. 9 can be applied to all sections starting from FIG. 3 up to FIG. 8.

The plates 3f and 3g may comprise a plurality of holes 9 configured to allow the passage of the sterilising fluid.

FIG. 10 shows a gap 6 comprising one or more diversion elements 7 configured to convey the circulation of the flow of sterilising fluid along a plurality of channels 10 internal to the gap 6.

Each internal channel 10 is separated from one another and has a mouth with greater flow rate, at each nozzle 4a, 4b, a restricted central zone and a terminal zone with greater flow rate again, in such a way as to favour a sort of venturi effect to the flow of fluid and to optimize the effectiveness of the ejector.

There may be one or more lateral partitions 8, perpendicular to the plate 3, configured to laterally close the gap 6.

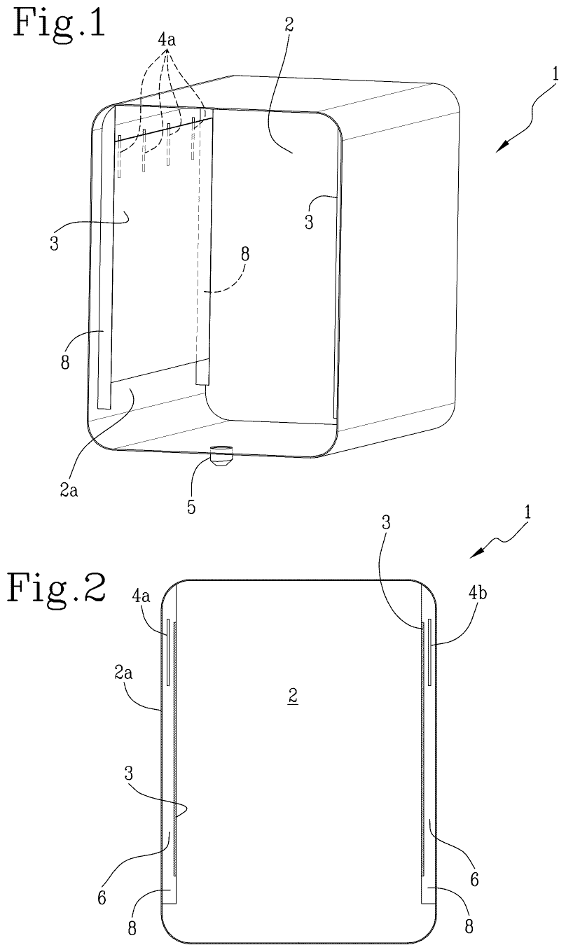

In a first embodiment, the nozzles 4a, 4b are with vertical axis, oriented vertically downwards of the sterilisation chamber 2 (FIGS. 1, 2 and 4-10).

In this case, the plates 3 are advantageously distanced by 40-60 mm from the respective internal walls of the chamber 2.

In a second embodiment, the nozzles 4a, 4b have an inclination with respect to the vertical axis of the sterilisation chamber 2 comprised between 10° and 110°, preferably comprised between 40° and 80°.

The nozzles 4a, 4b can be positioned distanced from one another, at about half the height of each plate 3, i.e. the nozzles 4a, 4b are positioned half way along the distance between the lower part and the upper part of the plate 3.

In a constructional variant of the second embodiment, the nozzles 4a, 4b can be positioned distanced from one another, in proximity to the upper edge of the surface of each plate 3.

In the variants of the second embodiment, the plates 3 are advantageously distanced by 30 mm from the respective internal walls of the chamber 2.

In principle, each ejector will consist of an assembly of plate 3, 3a-3g, plus nozzle 4a, 4b.

The position of the nozzles 4a, 4b may vary in height from a minimum of a few tens of millimetres from the upper edge of the plate 3 to a maximum of about half height of the plate 3. As a result, the shapes of the plate and flow deflectors will change.

The inclination and the direction of the flow of the nozzles 4a, 4b will tend to be vertical and parallel to the plate and to the wall of the autoclave.

In one configuration the nozzle 4a, 4b is inclined by about 30° on the vertical and directed against the plate 3.

In the lower part of the sterilisation chamber, e.g. in the lower surface, parallel to the floor on which the sterilisation machine rests, there is a vent 5.

Preferably, the vent 5 is positioned, at the bottom, in a central position.

It is also possible to have an additional vent 5 positioned on the ceiling of the sterilisation chamber. In this case, since the sterilisation gas (e.g. steam) is lighter than the air, the emptying of the gas present inside the sterilisation chamber is accelerated.

In this way the gas is sucked in (like in a hood) reducing the condensates present on the load. Any condensates that may form will be sucked with the vent 5 positioned at the bottom of the sterilisation chamber.

Preferably, the sterilising fluid is water vapour.

In a second aspect, the present invention describes a sterilisation method comprising the steps of:
introducing the products to be sterilised into a sterilisation chamber (2) of a sterilisation autoclave (1) through a passage;
hermetically closing the sterilisation chamber (2) with a movable door;
introducing a flow of sterilising fluid directly into an open gap (6) present in the sterilisation chamber (2), delimited by a plate (3) distanced from one of the internal walls of the sterilisation chamber (2), by one or more nozzles (4a, 4b), so that said flow of sterilising fluid introduced into the gap (6) creates a suction dragging effect in said secondary volume of fluid present inside the remaining volume of the sterilisation chamber (2) ensuring fluid circulation in said sterilisation chamber (2).

The present invention achieves the following technical effects:
a faster and more uniform distribution of the temperature inside the sterilisation chamber 2;
a faster and more uniform distribution of the sterilising fluid inside chamber 2;
energy saving, with the same sterilising power of the machine;
a decrease in the condensed mass of water;
the plates 3 and the position of the injectors 4a, 4b allow carrying out a circulation of the steam inside the sterilisation chamber 2, intensifying as much as possible the venturi effect in the channel 6 between the plates 3 and the internal wall of the chamber;
the presence of a channel between the plate and the internal wall is effective in carrying out an internal circulation, thanks to the positioning of the injectors, without the use of moving components;
when the inlet and outlet flow rate is zero, a natural convective motion is triggered inside the chamber, generated by the thermal flow to the outside and by the plates kept at a constant temperature;
even in these conditions the channel between the plate and the internal wall acts as a flow accelerator, exploiting the venturi effect, which could therefore be optimized even in a zero flow condition;
maintaining the plate at a high temperature allows local heating of the steam tending to rise in the part of the chamber inside the plates;
the amount of liquid (condensed steam) is very low and tends to accumulate in the lower part of the autoclave.

The invention as it is conceived is susceptible to numerous modifications and variants, all falling within the scope of the inventive concept characterised thereby. Furthermore, all of the details can be replaced with other technically equivalent elements. In practice, all the materials used, as well as the dimensions, can be any whatsoever, according to need.

1. A sterilisation autoclave (1) comprising:
a closed sterilisation chamber (2) intended to contain the products to be sterilised, said chamber (2) identifying an internal compartment and being provided with an opening that identifies a passage for inserting/collecting the products to be sterilised;
a removable door associated when closed with said opening for passing from an open position, distanced from said opening, to a closed position in which it guarantees the hermetic closure of said passage of said sterilisation chamber (2);
a plate (3) having two opposing sides with a greater extension and positioned in said sterilisation chamber (2) in proximity to a first wall (2a) of said sterilisation chamber (2), said plate (3) being positioned so as to have a first side (2a) of said two opposing sides facing said first wall (2a) and
one or more nozzles (4a, 4b) configured to introduce a flow of sterilising fluid into said sterilisation chamber (2),
wherein:
said plate (3) identifies with said first wall (2a) an open gap (6) having a predefined separate secondary volume and in fluid communication with the remaining volume of the sterilisation chamber (2),
said one or more nozzles (4a, 4b) are positioned to introduce a flow of sterilising fluid directly into said open gap (6), so that said flow of sterilising fluid introduced into the gap (6) creates a suction dragging effect into said secondary volume of fluid present inside the remaining volume of the sterilisation chamber (2) ensuring fluid circulation in said sterilisation chamber (2).

2. The autoclave according to claim 1, wherein inside the sterilisation chamber (2) there are two plates (3) parallel to one another, positioned at a distance from respective opposite walls of the sterilisation chamber (2), each plate (3) defining an open gap (6), in which there are one or more nozzles (4a, 4b).

3. The autoclave according to claim 2, wherein each plate (3) is distanced from an internal wall of the sterilisation chamber (2) between 10 mm and 100 mm, preferably between 30 mm and 50 mm.

4. The autoclave according to claim 3, wherein at least one plate (3) is:
flat;
convergent/divergent;
composed of two flat plates (3a, 3b), a first plate (3a) positioned close to the internal wall of the chamber (2) and a second plate (3b) distanced from said first plate (3a);
composed of a plurality of flat elements (3c, 3b, 3e) positioned so as to define one or more passages for the flow of fluid between the secondary volume of the gap (6) and the remaining volume of the chamber (2).

5. The autoclave according to claim 4, wherein said plate (3, 3b) comprises at one end a perforated deflecting baffle plate (3f).

6. The autoclave according to claim 4, wherein said plate (3, 3b) comprises at one end a perforated horizontal plate (3g).

7. The autoclave according to claim 4, wherein inside the gap (6) there are one or more diversion elements (7) configured to convey the circulation of the flow of sterilising fluid along a plurality of channels (10) internal to the gap (6).

8. The autoclave according to claim 1, where there are one or more lateral partitions (8), perpendicular to said plate (3), configured to laterally close the gap (6).

9. The autoclave according to claim 1, wherein the longitudinal axis of the nozzles (4a, 4b) is vertical.

10. The autoclave according to claim 1, wherein the longitudinal axis of the nozzles (4a, 4b) has an inclination with respect to the vertical axis of the sterilisation chamber (2) comprised between 10° and 110°, preferably comprised between 40° and 80°.

11. The autoclave according to claim 1, wherein the nozzles (4a, 4b) are positioned half way along the distance between a lower part and an upper part of each plate (3).

12. The autoclave according to claim 1, wherein the nozzles (4a, 4b) are positioned in the upper part of the plate (3).

13. The autoclave according to 1, wherein the at least one plate (3) identifies a coil (9) consisting of a tubular passage to let a working fluid flow therein, said coil (9) comprising an inlet end (10) and an outlet end (10) of the fluid.

14. The autoclave according to claim 1, wherein the at least one plate (3) comprises an electrical coil configured to convert electrical energy into thermal energy.

15. The autoclave according to claim 1, wherein the section of the autoclave (1) is cylindrical or quadrangular.

16. The autoclave according to claim 1, wherein said sterilising fluid is water vapour.

17. The autoclave according to claim 1, wherein said plate (3, 3a, 3b, 3c, 3d, 3e, 3f, 3g) is heated/cooled by a process fluid or other system.

18. A sterilisation method comprising the steps of:
introducing the products to be sterilised into a sterilisation chamber (2) of a sterilisation autoclave (1) through a passage;
hermetically closing the sterilisation chamber (2) with a movable door;
introducing a flow of sterilising fluid directly into an open gap (6) present in the sterilisation chamber (2), delimited by a plate (3) distanced from one of the internal walls of the sterilisation chamber (2), by one or more nozzles (4a, 4b), so that said flow of sterilising fluid introduced into the gap (6) creates a suction dragging effect in said secondary volume of fluid present inside the remaining volume of the sterilisation chamber (2) ensuring fluid circulation in said sterilisation chamber (2).
